(19) United States
(12) Patent Application Publication
(10) Pub. No.: US 2009/0133157 A1
(43) Pub. Date: May 21, 2009

(54) METHOD OF PRODUCING FRUIT OF CAPSICUM PLANT WITH VITAMIN C CONTENT INCREASED

(75) Inventor: Shinya Tsuda, Tsukuba-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Incorp Adm Agency Nat'l Agr & Food Res Org, Tsukuba-shi (JP)

(21) Appl. No.: 12/273,966

(22) Filed: Nov. 19, 2008

(30) Foreign Application Priority Data
Nov. 21, 2007 (JP) 2007-301377

(51) Int. Cl. A01H 5/08; C12N 15/82; A01H 5/00

(52) U.S. Cl. 800/278; 800/317.1

(57) ABSTRACT

The object of the present invention is to provide a method of producing a fruit of a Capsicum plant in which a vitamin C content, in particular a vitamin C content alone, is increased according to a common cultivating procedure without requiring special facility and cultivating procedure. The present invention provides a method of producing a fruit of a Capsicum plant with an increased vitamin C content, characterized by inoculating an attenuated strain of a virus belonging to Tomamovirus to a seedling of the Capsicum plant and cultivating the seedling to the Capsicum plant.

### BACKGROUND OF THE INVENTION

1. Field of the Invention

The present invention relates to a method of producing a garden crop with an improved function. More specifically, the present invention relates to a method of producing a fruit of a Capsicum plant with an increased vitamin C content by inoculating an attenuated strain of a virus belonging to Tobamovirus.

2. Description of the Related Art

With a boom of healthy foods in recent years, development of a food having an additionally improved functionality of the crop is desired.

The technology of applying a microorganism not having phytopathogenicity to the agriculture has been researched and developed as one of disease and insect-controlling technologies.

In view of the foregoing, a large number of useful microorganisms and attenuated strains of viruses exhibiting no pathogenicity against plants have been developed, and applied for patents so far.

However, the analysis of the food component in the harvest obtained by inoculating those non-pathogenic useful microorganisms and attenuated strains of viruses to the crop is, at the moment, limited to a method using a cucumber mosaic virus as described in JP 3133605 B.

JP 3133605 B describes an invention relating to a technology of increasing only vitamin C content of crops, which mainly targets the tomato, by using a cucumber mosaic virus classified into Cucumovirus. In addition to tomato, there are given, as applicable vegetables, cabbage, Chinese cabbage, radishes, cucumber, eggplant, asparagus, udo, spinach, watermelon, melon, peach, apple, and citrus.

By the way, of Capsicum plants, several tens of kinds are distributed over the world and cultivars exceeding several hundreds exist. A large number of those Capsicum plants contain capsaicin as a pungent component, but there are some cultivars having suppressed pungency, such as green pepper and paprika. In particular, the green pepper is used in a lot of foods, and one of the major crops in our country.

In addition, vitamin P which is contained with large amount in the Capsicum plant, particularly in the green pepper, may alleviate heat destruction of vitamin C. Therefore, with the green pepper, it is possible to ingest vitamin C much more than the lemon, for example. In other words, the Capsicum plant is extremely preferred crop for ingestion of vitamin C in terms of nutrition.

Accordingly, a fruit of the Capsicum plant is expected to be an extremely and nutritionally useful food for ingestion of vitamin C by increasing a vitamin C content, particularly, increasing only the vitamin C content in the fruit of the Capsicum plant.

However, there is no report that the cucumber mosaic virus increases the vitamin C content of the Capsicum plant at the present.

There is no description at all about Capsicum plants such as green pepper, chili pepper, paprika, and sweet pepper in JP 3133605 B.

Note that there is no report at the moment that the cucumber mosaic virus increases the vitamin C content of Capsicum plants in JP 3133605 B and even in other documents, while the case where the chili pepper and the green pepper are infected with the cucumber mosaic virus is known.

In addition, as methods of cultivating fruits and tomatoes, a cultivating method is reported in which the watering is controlled to be less in order to increase the sugar content of the fruit and the whole content of active ingredients other than sugar content is increased. However, special cultivating procedure and facility are required and in addition, it is unable to purely improve only the vitamin C content of the fruit.

Accordingly, the technology of increasing only the vitamin C content in the fruit of the Capsicum plant according to a common cultivating procedure without requiring special facility and a cultivating procedure is not established, and therefore, the development of the technology is expected.

### SUMMARY OF THE INVENTION

It is an object of the present invention to provide a method of producing a fruit of a Capsicum plant in which a vitamin C content, in particular a vitamin C content alone, is increased according to a common cultivating procedure without requiring special facility and a cultivating procedure, whereby solving the above-mentioned conventional problems.

The inventors of the present invention extensively studied for solving the above problems. As a result, the inventors found out that a fruit harvested after inoculating an attenuated strain of a virus belonging to Tomamovirus to a Capsicum plant had much more vitamin C content compared to uninoculated plants. The present invention is completed based on the findings.

That is, a first aspect of the present invention provides a method of producing a fruit of a Capsicum plant with an increased vitamin C content comprising inoculating an attenuated strain of a virus belonging to Tomamovirus to a seedling of the Capsicum plant and cultivating the seedling to the Capsicum plant.

A second aspect of the present invention provides a method of producing a fruit of a Capsicum plant according to the first aspect of the invention, in which the attenuated strain of virus belonging to Tomamovirus is an attenuated strain of Pepper mild mottle virus.

A third aspect of the present invention provides a method of producing a fruit of a Capsicum plant according to the first aspect or the second aspect of the invention, in which the Capsicum plant is Capsicum annuum.

A fourth aspect of the present invention provides a method of producing a fruit of a Capsicum plant according to the third aspect of the invention, in which the Capsicum annuum is a green pepper.

According to the present invention, it is possible to produce a fruit of a Capsicum plant in which a vitamin C content, in particular a vitamin C content alone, is increased according to a common cultivating procedure without requiring special facility and cultivating procedure.

That is, according to the present invention, the vitamin C content in the fruit of the Capsicum plant can be increased by inoculating an attenuated strain of a virus belonging to Tomamovirus to a seedling of the Capsicum plant and cultivating the seedling of the Capsicum plant according to a common cultivating procedure.

The attenuated strain of a virus belonging to Tomamovirus to be used in the present invention has a feature of being friendly to the environment and excellent in safety because the virus is obtained from the natural world, harmless to the human and the livestock, and does not have phytopathogenicity.

It is expected to provide a crop which matches with a recent health-consciousness because a high functional crop in which a vitamin C content, in particular a vitamin C content alone, is increased can be produced according to the present invention.

### BRIEF DESCRIPTION OF THE DRAWINGS

In the accompanying drawings:

FIG. 1 is a graph showing a vitamin C content of a harvested green pepper fruit in Example 4; and

FIGS. 2a, b, c and d are photographs showing growth forms of the plant and the form of the harvested fruit after cultivating in Example 4.

### DESCRIPTION OF THE INVENTION

Hereinafter, the present invention is described in detail.

The present invention relates to a method of producing a fruit of a Capsicum plant with an increased vitamin C content, characterized by inoculating an attenuated strain of virus belonging to Tomamovirus to a seedling of the Capsicum plant, and cultivating the seedling of the Capsicum plant.

Note that the vitamin C, content of which can be increased in the present invention, is L-ascorbic acid.

An attenuated strain of a virus maybe used as the virus belonging to Tomamovirus used in the present invention.

The attenuated strain is referred to as a strain which has no influence or slight influence on the growth of the Capsicum plant inoculated upon cultivating and includes a strain having slight influence to such an extent that a normal fruit can be produced. Preferred is a strain having no influence on the growth of the inoculated plant.

An attenuated strain that has been developed by a public institution may be used as the attenuated strain of a virus belonging to Tomamovirus in the present invention. Specifically, Pa 18 strain developed in Hokkaido Agricultural Research Center of The Ministry of Agriculture, Forestry and Fisheries of Japan, which is an attenuated strain of Pepper mild mottle virus (it is stored as an attenuated strain of Pepper mild mottle virus, MAFF No. 104086 strain, in National Institute of Agrobiological Sciences Genebank), C-1421 strain developed in Chiba-ken, and IPO-2-19 strain developed in Oita-ken may be used.

Note that the scientific name of the attenuated strain of Pepper mild mottle virus, MAFF No. 104086 strain stored in National Institute of Agrobiological Sciences Genebank is Pepper mild mottle virus (Tobamovirus) and the scientific name upon registration is Pepper mild mottle virus (Tobamovirus), which has phytopathogenicity but is not dangerous directly to the human body.

In addition, for example, as described in preparation examples below, an attenuated strain of virus which is suitable for cultivating characteristics may be selected by infecting a wild strain of a virus to a plant body and selecting a strain having no influence or slight influence on the growth form of the infected plant body.

Specifically, an attenuated strain of a virus, which is suitable for cultivating characteristics, may be selected by inoculating a wild strain of Pepper mild mottle virus stored in National Institute of Agrobiological Sciences Genebank (for example, MAFF NO. 104032 strain) to a Capsicum or Nicotiana plant, cultivating the plant under high temperature and a stressed condition, selecting the plant according to necrotic spots, and selecting a strain having no influence or slight influence on the growth form.

An attenuated strain No. 13 of Pepper mild mottle virus described later in Preparation example 1 is one of the attenuated strain of a virus obtained by the above-mentioned method.

Note that an attenuated strain of a virus which is suitable for cultivating characteristics may be selected by repeating the growth under high temperature and a stressed condition and selection according to the necrotic spot several times as required.

The scientific name of the wild strain of Pepper mild mottle virus, MAFF No. 104032 strain stored in National Institute of Agrobiological Sciences Genebank is Pepper mild mottle virus (Tobamovirus) and the scientific name upon registration is Tobacco mosaic virus (Tobamovirus), which has phytopathogenicity but is not dangerous directly to the human body.

As the attenuated strain of the virus belonging to Tobamovirus, an attenuated strain No. 13 of Pepper mild mottle virus described later in Preparation example 1 obtained by the above-mentioned method, may be used in the present invention. Because of being a virus, the No. 13 strain is not allowed to be deposited in any one of the International Patent Organism Depository of National Institute of Advanced Industrial Science and Technology and the Patent Microorganisms Depository of National Institute of Technology and Evaluation, which are assigned as deposit organizations by the commissioner of JPO. This attenuated strain may be deposited under the terms of the Budapest Treaty if required.

In addition, an attenuated strain No. 16 of Pepper mild mottle virus described in Preparation example 2 below may be used as an attenuated strain of the virus belonging to Tomamovirus in the present invention. The No. 16 strain is a virus as the No. 13 strain above, and No. 16 strain is not allowed to be deposited in any one of the International Patent Organism Depository of National Institute of Advanced Industrial Science and Technology and the Patent Microorganisms Depository of National Institute of Technology and Evaluation, which are assigned as deposit organizations by the commissioner of JPO. This attenuated strain may be deposited under the terms of the Budapest Treaty if required.

The kind of the attenuated strain of virus belonging to Tomamovirus to be used in the present invention may be determined appropriately by taking the cultivar of an objective Capsicum plant and purpose of use into consideration.

Note that the vitamin C content of the fruit can be increased in the present invention even in a case where a wild strain of a virus is used instead of attenuated strain. However, it is not preferred because the pathology by the infection is expressed in growth form; the number of fructification is decreased due to growth inhibition, and deformed fruit and mosaic fruit are generated, with the result that the deliverable harvest amount is extremely decreased and the economical efficiency is impaired.

The viruses belonging to Tomamovirus to be used in the present invention are different from the cucumber mosaic virus classified into Cucumovirus in terms of virology. The viruses are different largely in properties such as the virus particle form, the genomic structure, the mediation manner, and the range of plant to which the virus infects.

In addition, both are different particularly in the proliferation mechanism from the viewpoint of virus multiplication. Further, both are different in the resistance gene of the infected plant serving as a host and the responsive reaction involved in symptoms expression after infection of the virus.

As the virus belonging to Tomamovirus which can be used in the present invention, any virus is used as long as the Capsicum plant is infected with the virus, and the virus has an influence of increasing the vitamin C content of the fruit. Specific examples include Pepper mild mottle virus (PMMoV), Tobacco mosaic virus (TMV), and Tomato mosaic virus (ToMV). In particular, Pepper mild mottle virus (PMMoV) is preferably used in terms of relation with the resistance gene of the virus belonging to Capsicum.

Note that the virus belonging to Tomamovirus is a plant virus which infects only specific plants, such as plants belonging to Solanaceae including Capsicum, plants belonging to Cucurbitaceae, and plants belonging to Liliaceae, and is perfectly harmless to the human, livestock, and wild animals.

In addition, because the virus belonging to Tomamovirus to be used in the present invention is an attenuated strain, the pathology due to the infection does not influence on the growth form of the host plant.

In particular, Pepper mild mottle virus mainly used in the present invention is a virus which infects only a Capsicum plant. Further, because the proliferation amount of the attenuated strain in the infected plant body is about 20% to 30% compared to the wild strain, there is no possibility of influencing the plants in the open-field and other crops.

Accordingly, the present invention is a technology excellent in influence on the environment and the safety of foods.

In the present invention, the vitamin C content of the fruit of a plant having such a characteristics as classified into Capsicum can be increased by inoculating the attenuated strain of a virus belonging to Tomamovirus into or onto the seed, seedling, or plant, preferably into the seedling of Capsicum.

Of Capsicum plants, several tens of kinds are distributed over the world and cultivars exceeding several hundreds exist. Of those, a large number of Capsicum plants contain capsaicin as a pungent component.

Examples of the plant classified into Capsicum, which can be used in the present invention, include Capsicum annuum (capsicum in a broad sense), Capsicum baccatum (aji amarillo), Capsicum cardenasii (ulupica), Capsicum chinense Jacq. Heser & Smith (the similar member of habanero), Capsicum frutescens (bird pepper—material of tabasco), and Capsicum pubescens Ruiz & Rav. (rocoto).

The present invention is suited to increase the vitamin C content particularly of the fruit of Capsicum annuum (capsicum in a broad sense).

Here, examples of the Capsicum annuum (capsicum in a broad sense) include major crops such as green pepper, chili pepper, paprika, sweet chili pepper, Hontaka (a cultivar of the chili pepper), Jalapeno, and Takanotsume (a cultivar of the chili pepper), which are important food. Note that those crop cultivar are biologically belonging to the same kind (in which there is no reproductive isolation and the crossing is possible). Those are genetically assortative, which corresponds to the relationship between Koshihikari and Sasanishiki of the rice.

Of those, the green pepper, the chili pepper, the paprika, and the sweet chili pepper are preferred in the present invention, and the green pepper is most preferred. Accordingly, the present invention is most suited to increase the vitamin C content of the green pepper fruit.

The fruit of the Capsicum plant is referred to as ovary which is swelled after the floral organ is pollinated and contains the next-generation seed. The cavity accounts for most inside of the fruit, except for the seed, the placenta, and the dissepiment, and therefore, fleshy fruit is eaten. According to the present invention, specifically, vitamin C contained in the tissue of edible pulp part can be increased.

Note that a large amount of vitamin P is contained in the Capsicum plant, in particular, the green pepper. The vitamin P may alleviate heat destruction of vitamin C. Therefore, by eating a Capsicum plant, it is possible to ingest vitamin C much more than the lemon, for example. That is, the Capsicum plant is extremely suitable crop to ingest vitamin C in terms of nutrition.

The present invention is based on the findings that the vitamin C content of the fruit of the Capsicum plant can be increased by inoculating the virus belonging to Tomamovirus to the seedling of the Capsicum plant and infecting the seedling of the Capsicum plant with the virus. In particular, the present invention has effects of increasing the vitamin C content alone and not influencing contents of the other components.

As a method of increasing the sugar content, a conventional technology reported a cultivating procedure of increasing the whole contents of active ingredients (including vitamin C and the like) in addition to sugar content by controlling the watering to be less in order to increase the sugar content of the fruit. However, in this case, there were problems that special cultivating procedure and facility were required and only the vitamin C content of the fruit could not be purely improved.

With the present invention, it is possible to increase only the vitamin C content of the fruit of the Capsicum plant according to a common cultivating procedure without requiring the above special facility and cultivating procedure.

Note that the common cultivating procedure in the present invention is referred to as a soil culture cultivating using the soil and plant nutrition which is required to cultivate crops in the garden cultivating field. For example, with the method of the present invention, crops can be bred according to the general customary cultivating procedure practiced in many areas of the country where green peppers are produced such as Ibaraki-ken.

In other words, in the present invention, the control of the cultivation such as the raising of seedling, fertilization, and chemical spray may be performed according to the general control method of cultivating depending on the objective crop.

In the present invention, the attenuated strain of the virus belonging to Tomamovirus is specifically inoculated to the Capsicum plant as follows.

First, a leaf of the plant infected with the attenuated strain of the virus belonging to Tomamovirus is ground in a phosphate buffer solution using a mortar and a pestle. In a case where about 1 g of leaf is used, it is preferable to produce about 0.01 to 10 L, preferably about 1 L, of homogenates of phosphate buffer solution. As the leaf of the plant infected with the attenuated strain of the virus belonging to Tobamovirus, for example, a leaf of the a Nicotiana plant or a Capsicum plant, specifically a leaf of Nicotiana benthamiana may be used.

In addition, a phosphate buffer solution having a concentration of about 0.05 M and pH of about 7.0 may be used.

The period when the attenuated strain of the virus belonging to Tomamovirus is inoculated to a seedling of the Capsicum plant is preferably a seedling stage from the viewpoint of infectiosity. However, the attenuated strain can be inoculated at another stage.

Specific inoculation period is on the 10th to 20th day, and preferably the about 14th day after seeding of the Capsicum plant. At that time, the virus contained in the above homogenates is inoculated to a seedling, from which cotyledon is fully developed.

As the inoculation method, a polishing agent is sprinkled over the cotyledon and the cotyledon is rubbed softly with an absorbent cotton or the like saturated with homogenates, whereby the virus is inoculated to the seedling. As the polishing agent, for example, about 400-mesh to 600-mesh carborundum™ may be used.

Subsequently, in the present invention, by cultivating the plant with the above-mentioned common method, the fruit of the Capsicum plant with increased vitamin C content is produced.

The virus may be inoculated to the seedling in the open-field or inside a greenhouse. In a case of inoculating performed inside a greenhouse, it is preferred to plant the plant in the open-field, in a house, a farm field, a planter, or the like after about 10 true leaves are grown in the greenhouse. Note that the cultivating with the common method is referred to as cultivating according to the general customary cultivating method as described above, for example.

By inoculating the virus to the seedling as mentioned above, the Capsicum plant after inoculation can provide a Capsicum plant body, the whole body of which is infected with the attenuated strain of the virus belonging to Tobamovirus.

The above Capsicum plant can produce a fruit in which a vitamin C content, in particular a vitamin C content alone, is increased without influencing mostly contents of the other components. The vitamin C content of said plant is greater than that of a control plant produced by a seedling not inoculated with said virus. A fruit produced by a plant after inoculation of a seedling with Tomamovirus may have an increased vitamin C content ranging from >0, 1, 2, 5, 10, 20, 30, 40, 50, 100, 150, 200, 250, 300% or more than a fruit produced by a plant obtained from an non-innoculated seedling. The range above includes all intermediate subranges and values.

Note that, specifically, in a case where a seedling is planted to a farm field predetermined days after Pepper mild mottle virus is inoculated to a seedling of the green pepper and bred for about 3 months according to a general customary cultivating procedure, the vitamin C content of the harvested fruit can be increased about 1.3 to 2.0 times compared to an untreated plant. In a case where the plant is bred for about 4 months, the vitamin C content of the harvested fruit can be increased about 1.4 to 2.0 times compared to an untreated plant.

Note that there is no substantial influence or no detrimental influence on contents of the other components such as vitamin E, niacin, α-carotene, β-carotene, a soluble dietary fiber, and an insoluble dietary fiber by the inoculation of the attenuated strain of the virus belonging to Tomamovirus in the present invention.

In this way, the fruit of the Capsicum plant with vitamin C content increased can be produced by inoculating the attenuated strain of the virus belonging to Tomamovirus to the seedling of the Capsicum plant and cultivating the plant according to the common method.

As described above, the fruit of the Capsicum plant produced according to the above-mentioned method is suitable crop to ingest vitamin C in terms of nutrition, so it is expected to make the Capsicum plant an extremely useful food in order to ingest vitamin C in terms of nutrition.

### EXAMPLES

Hereinafter, the present invention is detailed by way of examples, but not limited thereto.

### Preparation Example 1

### Preparation of Attenuated Strain No. 13 of Pepper Mild Mottle Virus

The attenuated strain of Pepper mild mottle virus to be used in the following examples was selected.

First, an infected plant tissue of the green pepper infected with a wild strain of Pepper mild mottle virus (MAFF No. 104032) stored in National Institute of Agrobiological Sciences Genebank was added with 50 times amount of phosphate buffer solution (0.1 M phosphate buffer solution at around neutral) and ground. Note that the MAFF No. 104032 strain is such a wild strain that the yield throughout a single-crop is sharply decreased by causing mosaic symptoms involving deformation on the true leaf near the growing point of the infected green pepper plant body, causing deformed and green streak mosaic in the lateral direction on the fruit, and suppressing the growth of the plant.

By using the homogenates of pepper leaves infected with a wild strain of virus, the virus was inoculated to a main stem of the seedling of the green pepper (cultivar: New tosa hikari) which was grown up to 8-10 true leaf stage. The virus was inoculated by sprinkling a polishing agent (about 400-mesh to 600-mesh carborundum™) over the main stem of the seedling and rubbing the main stem softly with an absorbent cotton saturated with the homogenates of pepper leaves infected with a wild strain of virus. After the virus inoculation, the seedling was grown for about 3 to 4 weeks under high temperature and a stressed condition of 37 to 40° C. in a growth chamber.

After the growth, only the main stem inoculated with the wild strain of virus was cut out, 100 times amount of phosphate buffer solution (0.1 M phosphate buffer solution at around neutral) was added thereto, and the stem was ground. The 5th to 8th true leaves of Nicotiana tabacum cv. Xanthi nc, which was grown up to 10 true leaf stage, were inoculated with the virus by using the homogenates of the stem. The virus was inoculated by sprinkling a polishing agent (about 400-mesh to 600-mesh carborundum™) over the leaves and rubbing the leaves softly with an absorbent cotton saturated with the homogenates of the stem.

A large number of necrotic spots were separated one by one, which were formed due to the virus infection on the inoculated leaf after a lapse of 3 or 4 days from the virus inoculation. About 50 μL of a phosphate buffer solution (0.1 M phosphate buffer solution at around neutral) was added to each necrotic spot, thereby to obtain a crude solution.

Each crude solution prepared from each necrotic spot was inoculated to the green pepper seedling at the cotyledon stage. The virus was inoculated by sprinkling a polishing agent (about 400-mesh to 600-mesh carborundum™) over the seedling and rubbing the seedling softly with the crude solution using a glass latch. After the virus inoculation, the seedling was grown for about 1 month constantly between 23 and 25° C. in a glass greenhouse.

After that, the grown green pepper plant body was investigated on symptoms. An attenuated strains of Pepper mild mottle virus were isolated from the green pepper strain having no virus symptoms by eliminating the green pepper strain in which abnormal growth was recognized (infection of the wild strain) and the green pepper stain not infected with virus. The attenuated strain of Pepper mild mottle virus was defined as No. 13 strain.

Being a virus, the No. 13 strain was not allowed to be deposited in any one of the International Patent Organism Depository of National Institute of Advanced Industrial Science and Technology and the Patent Microorganisms Depository of National Institute of Technology and Evaluation, which are assigned as deposit organizations by the commissioner of JPO. Attenuated strain No. 13 of Pepper mild mottle virus may be deposited under the terms of the Budapest Treaty if required.

A leaf of the green pepper infected with the No. 13 strain selected by the above operation was added with 50 times amount of a phosphate buffer solution (0.1 M phosphate buffer solution at around neutral) and ground. Thus obtained homogenates of No. 13-infected pepper leaves was inoculated to the 5th to 8th leaves of Nicotiana benthamiana which was grown up to 10 true leaf stage. The virus was inoculated by sprinkling a polishing agent (about 400-mesh to 600-mesh carborundum™) over the leaves and rubbing the leaves softly with an absorbent cotton saturated with the homogenates of No. 13-infected pepper leaves. The inoculated leaves after a lapse of 5 days from the virus inoculation were collected and used as a leaf of Nicotiana benthamiana (inoculation source) infected with the attenuated strain of Pepper mild mottle virus (No. 13 strain) in the following examples. Preparation example 2 (Preparation of attenuated strain No. 16 of Pepper mild mottle virus) A plant tissue of the green pepper infected with a wild strain of virus was sampled at an area where green peppers are produced in Ibaraki-ken in 1996. Note that the wild strain of virus is such a strain of wild virus, as MAFF No. 104032 strain used in Preparation example 1, that the yield throughout a single-crop is sharply decreased by causing mosaic symptoms involving deformation on the true leaf near the growing point of the infected green pepper plant body, causing deformed and green streak mosaic in the lateral direction on the fruit, and suppressing the growth of the plant.

In Preparation example 2, an attenuated strain of Pepper mild mottle virus to be used in the following examples was isolated in the same way as in Preparation example 1 except that the tissue of the plant infected with the wild strain of virus sampled at an area where green peppers are produced in Ibaraki-ken in 1996 was used.

The attenuated strain of Pepper mild mottle virus selected in Preparation example 2 was defined as No. 16 strain. An inoculated leaf of Nicotiana benthamiana after a lapse of 5 days from the inoculation with the attenuated strain of Pepper mild mottle virus (No. 16 strain) was used in the following examples.

The No. 16 strain is a virus as the No. 13 strain above, the No. 16 strain was not allowed to be deposited in any of the International Patent Organism Depository of National Institute of Advanced Industrial Science and Technology and the Patent Microorganisms Depository of National Institute of Technology and Evaluation, which are assigned as deposit organizations by the commissioner of JPO. Attenuated strain No. 16 of Pepper mild mottle virus will be deposited under the terms of the Budapest Treaty if required.

### Example 1

1 g of the leaf of Nicotiana benthamiana infected with the attenuated strain of Pepper mild mottle virus (No. 13 strain) obtained in Preparation example 1 was ground in IL of 0.05 M phosphate buffer solution (pH 7.0) using a mortar and a pestle, to thereby prepare homogenates of No. 13-infected leaves.

In addition, 1 g of the leaf of Nicotiana benthamiana infected with a wild strain of virus (MAFF NO. 104032 strain) was ground in 1 L of 0.05 M phosphate buffer solution (pH 7.0) using a mortar and a pestle, to thereby prepare homogenates of wild strain-infected leaves.

Next, a seed of the green pepper (cultivar: New tosa hikari (Nangoku seed)) was seeded (Feb. 21, 2006). The virus was inoculated to the seedling by sprinkling a polishing agent (about 400-mesh to 600-mesh carborundum™) over cotyledon developing completely after a lapse of 14 days from the seeding and rubbing the cotyledon softly with an absorbent cotton saturated with either homogenates.

Then, after grown in a greenhouse until about 10 true leaves were grown, the seedling was planted to a protected field in KOIBUCHI College of Agriculture in Mito-shi, Ibaraki-ken (Apr. 7, 2006), to thereby cultivate the plant. Note that the control of the cultivation such as the raising of seedling, fertilization, and chemical spray may be performed according to the general control method of cultivating.

After a lapse of about 4 months from the planting (Aug. 10, 2006), fruits of the grown green pepper were harvested, thereby to measure the contents of vitamin C, vitamin E, niacin, α-carotene, β-carotene, a soluble dietary fiber, and an insoluble dietary fiber. The contents of vitamin C, vitamin E, α-carotene, and β-carotene were measured with HPLC, the content of niacin was measured by a microbiological assay, and the contents of the soluble dietary fiber and the insoluble dietary fiber were measured by Prosky method (“New food analysis method” edited by Japanese society for food science and technology). The contents were measured for 100 g of fruit.

Note that, for comparing with standard crops, Table 1 shows the value quoted from Standard tables of food composition in Japan fifth revised and enlarged edition.

Of those harvested green pepper fruits, a fruit harvested from a plant inoculated with the attenuated virus was defined as Present Invention Product 1, a fruit harvested from a plant inoculated with the wild virus as Comparative Product 1, and a fruit harvested from a plant not inoculated with virus as Comparative Product 2. Table 2 shows results.

TABLE 1

| | Value of Standard tables of food | |
|---|---|---|
| | composition in Japan fifth revised and | |
| | enlarged edition (per 100 g) | |
| Vitamin C (mg) | 76.0 | (mg) |
| Vitamin E (mg) | 0.8 | (mg) |
| Niacin (mg) | 0.6 | (mg) |
| α-carotene (μg) | 6.0 | (μg) |
| β-carotene (μg) | 400.0 | (μg) |
| (Soluble) Dietary fiber (g) | 0.6 | (g) |
| (Insoluble) Dietary fiber (g) | 1.7 | (g) |
| Dietary fiber (Total amount) (g) | 2.3 | (g) |

TABLE 2

| | Present | | | | | |
|---|---|---|---|---|---|---|
| | Invention | | | | | |
| | Product 1 | | Comparative | | Comparative | |
| | (inoculated | | Product 1 | | Product 2 | |
| | with | | (inoculated | | (not | |
| | attenuated | | with wild | | inoculated | |
| | virus) | | virus) | | with virus) | |
| Vitamin C (mg) | 130.0 | (mg) | 120.0 | (mg) | 93.0 | (mg) |
| Vitamin E (mg) | 0.6 | (mg) | 0.6 | (mg) | 0.5 | (mg) |
| Niacin (mg) | 0.8 | (mg) | 0.7 | (mg) | 0.6 | (mg) |
| α-carotene (μg) | 8.0 | (μg) | 6.0 | (μg) | 5.0 | (μg) |
| β-carotene (μg) | 290.0 | (μg) | 380.0 | (μg) | 310.0 | (μg) |
| (Soluble) Dietary fiber | 0.5 | (g) | 0.1 | (g) | 0.4 | (g) |
| (g) | | | | | | |
| (Insoluble) Dietary fiber | 1.8 | (g) | 1.8 | (g) | 1.7 | (g) |
| (g) | | | | | | |
| Dietary fiber (Total | 2.3 | (g) | 1.9 | (g) | 2.1 | (g) |
| amount) (g) | | | | | | |

| | | | |
|---|---|---|---|
| Influence on growth form | Not influenced | Influenced | Uninoculation |
| Period from planting | About 4 | About 4 | About 4 |
| to harvest | months | months | months |

As shown in Table 2, by inoculating the attenuated strain of Pepper mild mottle virus (No. 13 strain) to a seedling of the green pepper and cultivating the plant for about 4 months from the planting according to the common method (Present Invention Product 1), the vitamin C content of the fruit was increased 1.4 times compared to the case of uninoculated plants (Comparative Product 2). Note that there was no detrimental or substantial difference in contents of vitamin E, niacin, α-carotene, β-carotene, the soluble dietary fiber, and the insoluble dietary fiber, which were measured as contents of the other components. In addition, there was no influence on the growth form by the virus inoculation.

In addition, in a case where the wild strain of virus was inoculated (Comparative Product 1), the result was the same as in Present Invention Product 1 inoculated with the attenuated strain of virus with respect to the effect of increasing only the vitamin C content of the fruit. However, in a case of Comparative Product 1, the growth of the plant was suppressed, and the number of fructification was decreased, and further, the influence on the growth form caused by the virus infection (abnormal fruits such as deformed fruit and mosaic fruit) was observed.

### Example 2

1 g of the leaf of Nicotiana benthamiana infected with the attenuated strain of Pepper mild mottle virus (No. 16 strain) obtained in Preparation example 2 was ground in 1L of 0.05 M phosphate buffer solution (pH 7.0) using a mortar and a pestle, to thereby prepare homogenates of No. 16-infected leaves. In addition, 1 g of the leaf of Nicotiana benthamiana infected with a wild strain of virus (wild strain sampled at an area where green peppers are produced in Ibaraki-ken in 1996) was ground in 1 L of 0.05 M phosphate buffer solution (pH 7.0) using a mortar and a pestle, to thereby prepare homogenates of wild strain-infected leaves.

Next, a seed of the green pepper (cultivar: Miogi (Japan Horticultural Production and Research Institute)) was seeded (Feb. 20, 2006). The virus was inoculated to the seedling by sprinkling a polishing agent (400-mesh to 600-mesh carborundum™) over cotyledon developing completely after a lapse of 14 days from the seeding and rubbing the cotyledon softly with an absorbent cotton saturated with either homogenates.

Then, after grown in a greenhouse until about 10 true leaves were grown, the seedling was planted to a field in KOIBUCHI College of Agriculture in Mito-shi, Ibaraki-ken (Apr. 7, 2006), to thereby cultivate the plant. Note that the control of cultivation, such as raising of seedling, fertilization, and chemical spray may be performed according to the general control method of cultivating.

After a lapse of about 3 months from the planting (Jul. 5, 2006), and after a lapse of about 4 months from the planting (Aug. 2, 2006) as well, each fruit of the grown green pepper was harvested, thereby to measure the contents of vitamin C, vitamin E, niacin, α-carotene, β-carotene, the soluble dietary fiber, and the insoluble dietary fiber. The contents of vitamin C, vitamin E, α-carotene, and β-carotene were measured with HPLC, the content of niacin was measured by a microbiological assay, and the contents of the soluble dietary fiber and the insoluble dietary fiber were measured by Prosky method (“New food analysis method” edited by Japanese society for food science and technology). The contents were measured for 100 g of fruit.

Of those harvested green pepper fruits, a fruit harvested from a plant inoculated with the attenuated virus after a lapse of about 3 months from the planting was defined as Present Invention Product 2, a fruit harvested from a plant inoculated with the wild virus as Comparative Product 3, and a fruit harvested from a plant not inoculated with virus as Comparative Product 4. Table 3 shows results.

In addition, of those harvested green pepper fruits, a fruit harvested from a plant inoculated with the attenuated virus after a lapse of about 4 months from the planting was defined as Present Invention Product 3, a fruit harvested from a plant inoculated with the wild virus as Comparative Product 5, and a fruit harvested from a plant not inoculated with virus as Comparative Product 6. Table 4 shows results.

TABLE 3

| | Present | | | | | |
|---|---|---|---|---|---|---|
| | Invention | | | | | |
| | Product 2 | | Comparative | | Comparative | |
| | (inoculated | | Product 3 | | Product 4 | |
| | with | | (inoculated | | (not | |
| | attenuated | | with wild | | inoculated | |
| | virus) | | virus) | | with virus) | |
| Vitamin C (mg) | 90.0 | (mg) | 91.0 | (mg) | 54.0 | (mg) |
| Vitamin E (mg) | 0.8 | (mg) | 0.7 | (mg) | 0.9 | (mg) |
| Niacin (mg) | 0.7 | (mg) | 0.6 | (mg) | 0.6 | (mg) |
| α-carotene (μg) | 13.0 | (μg) | 15.0 | (μg) | 24.0 | (μg) |
| β-carotene (μg) | 470.0 | (μg) | 650.0 | (μg) | 780.0 | (μg) |
| (Soluble) Dietary fiber | 0.1 | (g) | 0.1 | (g) | 0.2 | (g) |
| (g) | | | | | | |
| (Insoluble) Dietary fiber | 1.4 | (g) | 1.0 | (g) | 0.9 | (g) |
| (g) | | | | | | |
| Dietary fiber (Total | 1.5 | (g) | 1.1 | (g) | 1.1 | (g) |
| amount) (g) | | | | | | |

| | | | |
|---|---|---|---|
| Influence on growth form | Not influenced | Influenced | Uninoculation |
| Period from planting | About 3 | About 3 | About 3 |
| to harvest | months | months | months |

TABLE 4

| | Present | | | | | |
|---|---|---|---|---|---|---|
| | Invention | | | | | |
| | Product 3 | | Comparative | | Comparative | |
| | (inoculated | | Product 5 | | Product 6 | |
| | with | | (inoculated | | (not | |
| | attenuated | | with wild | | inoculated | |
| | virus) | | virus) | | with virus) | |
| Vitamin C (mg) | 100.0 | (mg) | 110.0 | (mg) | 66.0 | (mg) |
| Vitamin E (mg) | 0.8 | (mg) | 0.7 | (mg) | 0.8 | (mg) |
| Niacin (mg) | 0.6 | (mg) | 0.5 | (mg) | 0.6 | (mg) |
| α-carotene (μg) | 6.0 | (μg) | 10.0 | (μg) | 10.0 | (μg) |
| β-carotene (μg) | 410.0 | (μg) | 410.0 | (μg) | 470.0 | (μg) |
| (Soluble) Dietary fiber | 0.3 | (g) | 0.3 | (g) | 0.2 | (g) |
| (g) | | | | | | |
| (Insoluble) Dietary fiber | 1.6 | (g) | 1.1 | (g) | 1.2 | (g) |
| (g) | | | | | | |
| Dietary fiber (Total | 1.9 | (g) | 1.4 | (g) | 1.4 | (g) |
| amount) (g) | | | | | | |

| | | | |
|---|---|---|---|
| Influence on growth form | Not influenced | Influenced | Uninoculation |
| Period from planting | About 4 | About 4 | About 4 |
| to harvest | months | months | months |

As shown in Table 3, by inoculating the attenuated strain of Pepper mild mottle virus (No. 16 strain) to a seedling of the green pepper and cultivating the plant for about 3 months from the planting according to the common method (Present Invention Product 2), the vitamin C content of the fruit was increased 1.67 times compared to the case of an uninoculated plant (Comparative Product 4).

In addition, as shown in Table 4, by cultivating the plant for about 4 months from the planting according to the common method (Present Invention Product 3), the vitamin C content of the fruit was increased 1.52 times compared to the case of an uninoculated plant (Comparative Product 6).

Note that there was no substantial influence or no detrimental difference in contents of vitamin E, niacin, α-carotene, β-carotene, the soluble dietary fiber, and the insoluble dietary fiber, which were measured as contents of the other components. In addition, there was no influence on the growth form by the virus inoculation.

In addition, in cases where the wild strain of virus was used (Comparative Product 3 and Comparative Product 5) shown in Table 3 and Table 4, the results were the same as that with attenuated strain with respect to the effect of increasing only the vitamin C content of the fruit. However, in cases of Comparative Product 3 and Comparative Product 5, the growth of the plant was suppressed, and the number of fructification was decreased, and further, the influence on the growth form caused by the virus infection (abnormal fruits such as deformed fruit and mosaic fruit) was observed.

### Example 3

1 g of the leaf of Nicotiana benthamiana infected with the attenuated strain of Pepper mild mottle virus (No. 16 strain) obtained in Preparation example 2 was ground in 1 L of 0.05 M phosphate buffer solution (pH 7.0) using a mortar and a pestle, to thereby prepare homogenates of No. 16-infected leaves. In addition, 1 g of the leaf of Nicotiana benthamiana infected with a wild strain of virus (wild strain sampled at an area where green peppers are produced in Ibaraki-ken in 1996) was ground in 1 L of 0.05 M phosphate buffer solution (pH 7.0) using a mortar and a pestle, to thereby prepare homogenates of wild strain-infected leaves.

Next, a seed of the green pepper (cultivar: Miogi (Japan Horticultural Product and Research Institute)) was seeded (Feb. 20, 2006). The virus was inoculated to the seedling by sprinkling a polishing agent (400-mesh to 600-mesh carborundum™) over cotyledon developing completely after a lapse of 14 days from the seeding and rubbing the cotyledon softly with an absorbent cotton saturated with either homogenates.

Then, after grown in a greenhouse until about 10 true leaves were grown, the seedling was planted to a field in National Agricultural Research Center in Tsukuba-shi, Ibaraki-ken (Apr. 7, 2006), to thereby cultivate the plant. Note that the control of cultivation, such as raising of seedling, fertilization, and chemical spray may be performed according to the general control method of cultivating.

After a lapse of about 3 months from the planting (Jul. 5, 2006), and after a lapse of about 4 months from the planting (Aug. 2, 2006) as well, the fruit of the grown green pepper was harvested, thereby to measure the contents of vitamin C, vitamin E, niacin, α-carotene, β-carotene, the soluble dietary fiber, and the insoluble dietary fiber. The contents of vitamin C, vitamin E, α-carotene, and β-carotene were measured with HPLC, the content of niacin was measured by a microbiological assay, and the contents of the soluble dietary fiber and the insoluble dietary fiber were measured by Prosky method (“New food analysis method” edited by Japanese society for food science and technology). The contents were measured for 100 g of fruit.

Of those harvested green pepper fruits, a fruit harvested from a plant inoculated with the attenuated virus after a lapse of about 4 months from the planting was defined as Present Invention Product 4, a fruit harvested from a plant inoculated with the wild virus as Comparative Product 7, and a fruit harvested from a plant not inoculated with virus as Comparative Product 8. Table 5 shows results.

TABLE 5

| | Present | | | | | |
|---|---|---|---|---|---|---|
| | Invention | | | | | |
| | Product 4 | | Comparative | | Comparative | |
| | (inoculated | | Product 7 | | Product 8 | |
| | with | | (inoculated | | (not | |
| | attenuated | | with wild | | inoculated | |
| | virus) | | virus) | | with virus) | |
| Vitamin C (mg) | 110.0 | (mg) | 130.0 | (mg) | 73.0 | (mg) |
| Vitamin E (mg) | 0.9 | (mg) | 1.3 | (mg) | 0.7 | (mg) |
| Niacin (mg) | 0.5 | (mg) | 0.5 | (mg) | 0.4 | (mg) |
| α-carotene (μg) | 5.0 | (μg) | 10.0 | (μg) | 8.0 | (μg) |
| β-carotene (μg) | 370.0 | (μg) | 400.0 | (μg) | 540.0 | (μg) |
| (Soluble) Dietary fiber | 0.7 | (g) | 0.5 | (g) | 0.4 | (g) |
| (g) | | | | | | |
| (Insoluble) Dietary fiber | 1.6 | (g) | 1.7 | (g) | 1.6 | (g) |
| (g) | | | | | | |
| Dietary fiber (Total | 2.3 | (g) | 2.2 | (g) | 2.0 | (g) |
| amount) (g) | | | | | | |

| | | | |
|---|---|---|---|
| Influence on growth form | Not influenced | Influenced | Uninoculation |
| Period from planting | About 4 | About 4 | About 4 |
| to harvest | months | months | months |

As shown in Table 5, by inoculating the attenuated strain of Pepper mild mottle virus (No. 16 strain) to a seedling of the green pepper and cultivating the plant for about 4 months from the planting according to the common method (Present Invention Product 4), the vitamin C content of the fruit was increased 1.51 times compared to the case of an uninoculated plant (Comparative Product 8).

Note that there was no detrimental difference or no substantial difference in contents of vitamin E, niacin, α-carotene, β-carotene, the soluble dietary fiber, and the insoluble dietary fiber, which were measured as contents of the other components. In addition, there was no influence on the growth form by the virus inoculation.

In addition, in case where the wild strain of virus was used (Comparative Product 7), the result was the same as that with attenuated strain with respect to the effect of increasing only the vitamin C content of the fruit. However, in cases of Comparative Product 7, the growth of the plant was suppressed, and the number of fructification was decreased, and further, the influence on the growth form caused by the virus infection (abnormal fruits such as deformed fruit and mosaic fruit) was observed.

### Example 4

1 g of the leaf of Nicotiana benthamiana infected with the attenuated strain of Pepper mild mottle virus (No. 16 strain) obtained in Preparation example 2 was ground in 1 L of 0.05 M phosphate buffer solution (pH 7.0) using a mortar and a pestle, to thereby prepare homogenates of No. 16-infected leaves.

Next, a seed of the green pepper (cultivar: Miogi, Japan Horticultural Product and Research Institute) was seeded (Jan. 6, 2006). The virus was inoculated to the seedling by sprinkling a polishing agent (400-mesh to 600-mesh carborundum™) over cotyledon developing completely after a lapse of 14 days from the seeding and rubbing the cotyledon softly with an absorbent cotton saturated with homogenates of No. 16-infected leaves.

Then, after grown in a greenhouse until about 10 true leaves were grown, the seedling was planted to a field in National Agricultural Research Center in Tsukuba-shi, Ibaraki-ken (Jan. 22, 2007), to thereby cultivate the plant. Note that the control of cultivation, such as raising of seedling, fertilization, and chemical spray may be performed according to the general control method of cultivating.

After a lapse of about 4 months from the planting (May 17, 2007), the fruit of the grown green pepper was harvested, thereby to measure the content of vitamin C. The contents of vitamin C, vitamin E, α-carotene, and β-carotene were measured with HPLC, the content of niacin was measured by a microbiological assay, and the contents of the soluble dietary fiber and the insoluble dietary fiber were measured by Prosky method (“New food analysis method” edited by Japanese society for food science and technology). The content was measured for 100 g of fruit.

Of those harvested green pepper fruits, a fruit harvested from a plant inoculated with the attenuated virus was defined as Present Invention Product 5, and a fruit harvested from a plant not inoculated with virus as Comparative Product 9.

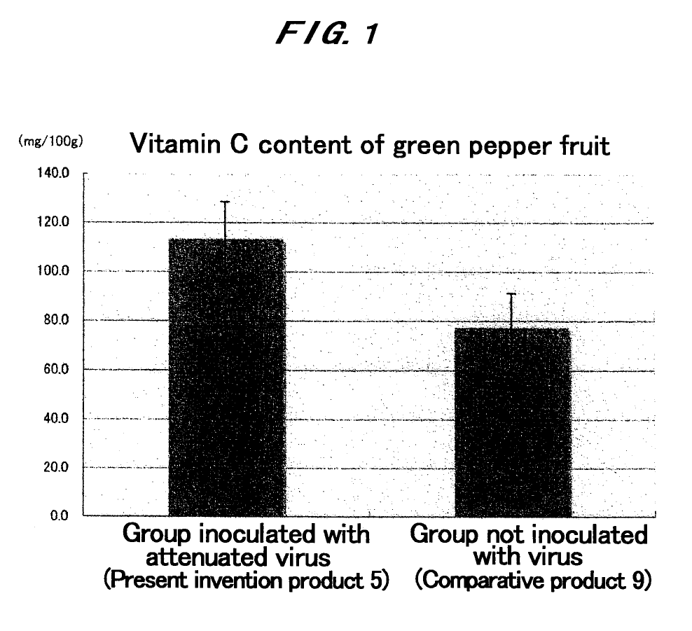

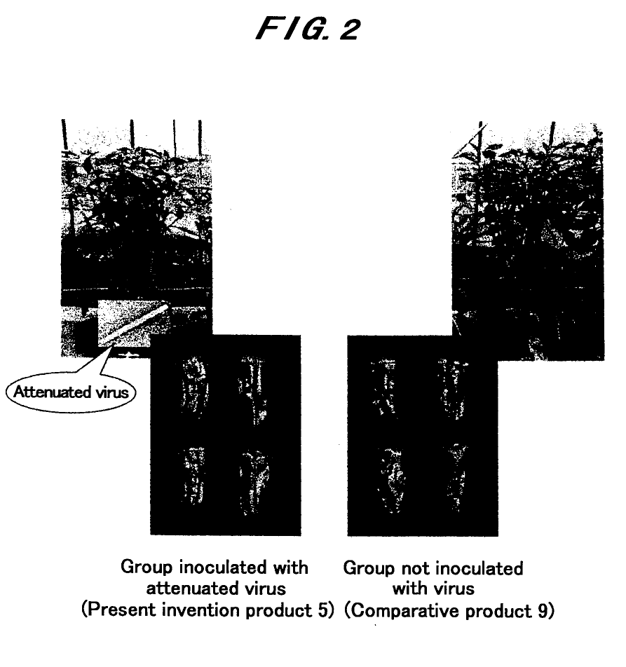

FIG. 1 shows the vitamin C content per 100 g of the harvested green pepper fruit. FIG. 2 shows the growth form of the plant body and the form of the harvested fruit after cultivating.

As shown in FIG. 1, by inoculating the attenuated strain of Pepper mild mottle virus (No. 16 strain) to a seedling of the green pepper and cultivating the plant for about 4 months from the planting according to the common method (Present Invention Product 5), the vitamin C content of the fruit was increased 1.46 times on an average compared to the case of an uninoculated plant (Comparative Product 9).

In addition, as shown FIG. 2, there was no influence on the growth form of the plant body and the form of the harvested fruit by the virus inoculation.

It is expected to provide a crop which matches with a recent health-consciousness because a high functional crop in which a vitamin C content, in particular a vitamin C content alone, is increased can be produced according to the present invention. Further, the present invention is a method friendly to the environment and excellent in safety in addition to that the present invention can be conducted according to a conventional cultivating procedure without requiring special facility and cultivating procedure, thereby expected to be widely applied to the agriculture field and food field.

What is claimed is:

1. A method of producing a fruit of a Capsicum plant with an increased vitamin C content, comprising:
inoculating an attenuated strain of a virus belonging to Tomamovirus to a seedling of the Capsicum plant; and
cultivating the seedling of the Capsicum plant into a fruit-producing plant.

2. A method of producing a fruit of a Capsicum plant according to claim 1, wherein the attenuated strain of the virus belonging to Tomamovirus is an attenuated strain of Pepper mild mottle virus.

3. A method of producing a fruit of a Capsicum plant according to claim 1, wherein the Capsicum plant is Capsicum annuum.

4. A method of producing a fruit of a Capsicum plant according to claim 3, wherein the Capsicum annuum is a green pepper.

5. The method of claim 1, further comprising harvesting a fruit having increased vitamin C content compared to a fruit harvested from a plant produced from an otherwise similar seedling not inoculated with said attenuated strain of a virus belonging to Tobamovirus.

6. The method of claim 1, wherein said fruit contains at least 1.3 times the amount of vitamin C compared to a fruit harvested from a plant produced from an otherwise similar seedling not inoculated with said attenuated strain of a virus belonging to Tobamovirus.

7. A fruit of a Capsicum plant with an increased vitamin C content produced by the method of claim 1.

8. The fruit of claim 7, which contains has at least 1.3 times the amount of vitamin C compared to a fruit harvested from a plant produced from an otherwise similar seedling not inoculated with said attenuated strain of a virus belonging to Tobamovirus.
